(19) United States
(12) Patent Application Publication
(10) Pub. No.: US 2011/0052549 A1
(43) Pub. Date: Mar. 3, 2011

(54) CELL CULTURE DEVICE TO DIFFERENTIATE STEM CELLS IN A SPECIFIC ORIENTATION

(75) Inventors: Wei-Chun Chin, Merced, CA (US); Tzu-I Chao, Merced, CA (US); Eric Yi-Tong Chen, Merced, CA (US); Chi-Shuo Chen, Merced, CA (US)

(73) Assignee: The Regents of the University of California

(21) Appl. No.: 12/869,616

(22) Filed: Aug. 26, 2010

Related U.S. Application Data
(60) Provisional application No. 61/237,651, filed on Aug. 27, 2009.

(51) Int. Cl. A61K 35/30; C12N 5/07; C12N 1/00; C12N 5/0735; C12N 5/071; A61K 35/12; C12N 5/0793; C12Q 1/02; A61P 25/28

(52) U.S. Cl. 424/93.7; 435/375; 435/243; 435/352; 435/363; 435/366; 435/325; 435/29

(57) ABSTRACT

Methods and compositions for aligning cell growth is provided by this invention by contacting at least one isolated cell with a device, wherein the device comprises a plurality of continuously orientating substantially parallel structures on the surface of the device, and culturing the cell under conditions that favor division of the at least one cell into a population of cells, thereby growing the population of cells in a substantially parallel orientation.

### CROSS-REFERENCE TO RELATED APPLICATION

This application claims the benefit under 35 U.S.C. §119(e) of U.S. Provisional Application No. 61/237,651, filed Aug. 27, 2009, the contents of which are hereby incorporated by reference into the present disclosure.

### BACKGROUND

Throughout this disclosure, various technical and patent publications are referenced to more fully describe the state of the art to which this invention pertains. These publications are incorporated by reference, in their entirety, into this application.

Stem cells are capable of self-renewal through numerous cycles of cell divisions and capable of transformation into specialized cell types. The ultimate cell type into which the stem cells will differentiate depends on intrinsic regulatory factors and the microenvironment. Lee et al. (2005) Tissue Eng. and Reg. Med. 2(3):264-273.

Stem cells are typically classified into several types: embryonic stem cells (ESCs) found in blastocysts, adult stem cells found in post-embryonic tissues and induced pluripotent stem cells (iPSCs) which have been de-differentiated from the adult type into an embryonic-type state. ESCs are extracted from the inner cell mass (ICM) of blastocysts within 14 days after fertilization. Adult stem cells act extracted from fetal and adult tissue and, for the most part, are limited in the cell types into which they can differentiate. Although they have limited differentiating capacity as compared to ESC, the adult stem cells can function stably and have been shown to differentiate across some tissue types. Pittenger et al. (1999) Science 284(2):143-147 and Huard et al. (2004) Curr. Opin. Biotechnol. 15(5):419-23.

Adult stem cells that can be utilized to treat nervous-system diseases are neural stem cells. However, because these stem cells exist in specific regions of the brain, such as the subventricular zone (SVZ) and the hippocampus, it is impossible to isolate them in therapeutically sufficient amounts. Bone marrow-derived mesenchymal stem cells, muscle-derived stem cells and adipose-derived stem cells are advantageous in that they exhibit in vitro self-renewing abilities and can be easily isolated and cultured as adult stem cells capable of differentiating into neurons, bones, cartilages and adipose tissues under adequate conditions for differentiation.

Embryonic stem cells also have been differentiated into neural cells but the techniques have been hampered by the inability to obtain homogenous populations and the glial cell subtype. Suter et al. (2008) J. of Path. 215(4):355-368.

Even with these limitations, neural stem and progenitor cells offer great potential for treatment of neurological disorders, e.g., Traumatic Brain Injury, Alzheimer's disease, Parkinson's disease, epilepsy, Huntington's disease, and stroke. Cell populations that can reconstitute the neural network is key to realizing these therapies.

Methods and compositions for differentiating adult and embryonic stem cell populations to neural cell populations are known in the art and described in U.S. Patent Publ. No. 2009/0035284 which itself teaches the differentiation of embryonic stem cells into a homogeneous population of neural stem cells with unlimited self-renewal capability. U.S. Pat. No. 7,011,828 and U.S. Patent Publ. Nos. 2005/0260747 A1 and 2006/0078543 A1 are reported to teach the proliferation of an enriched population of embryonic stem cells, which are induced to differentiate in vitro to neural progenitor cells, neurons, and/or glial cells. U.S. Pat. No. 6,887,706 teaches a method of differentiating embryonic stem cells into neural precursor cells using the growth factor FGF2. In vitro differentiation of the ES cell-derived neural precursors was induced by withdrawal of FGF2 and plating on ornithine and laminin substrate.

U.S. Pat. No. 7,015,037 and U.S. Patent Publication No. 2006/0030041 A1 are reported to teach the differentiation of multipotent adult stem cells (MASCs) to form glial, neuronal, or oligodendrocyte cell types using growth factors, chemokines, and cytokines such as EGF, PDGF-BB, FGF2, and FGF-9. U.S. Patent Publication No. 2006/0252149 is alleged to disclose the maintenance of central nervous system (CNS) cells in vitro that retain the ability to proliferate and remain in an undifferentiated state by culturing in the presence of soluble laminin alone or together with one or more laminin associated factors (LAFs) and one or more of the CNS mitogens EGF, bFGF (also referred to as FGF2), and LIF.

U.S. Patent Publ. No. 2007/0059823 A1 discloses a method for inducing ES cells and MASCs to differentiate into neuronal cells by culturing the stem cells initially with bFGF and later with FGF8, Sonic Hedgehog, brain-derived neutrotrophic factor, and astrocytes. Supplementing the media with growth factors such as EGF, platelet derived growth factor (PDGF), and LIF keeps the cells in an undifferentiated state.

U.S. Patent Publ. No. 2005/0214941 A1 teaches a method for improving the growth rate of human fetal brain stem cells by culturing human neural stem cells (hNSCs) with bFGF, EGF, and LIF. Culturing the hNSCs on a surface coated with polyornithine and fibronectin increases the rate of proliferation of neural stem cell cultures and increasing the number of neurons.

The technical literature also reports methods and compositions to culture neural stem cells. For example, Ling et al. (1998) Exp. Neurol. 149:411, reports the isolation of progenitor cells from the germinal region of rat fetal mesencephalon. The cells were grown in EGF and plated on poly-lysine coated plates, whereupon they formed neurons and glia, with occasional tyrosine hydroxylase positive (dopaminergic) cells, enhanced by including IL-1, IL-11, LIF, and GDNF in the culture medium.

Wagner et al. (1999) Nature Biotechnol. 17:653, reports the induction of cells with a ventral mesencephalic dopaminergic phenotype from an immortalized multipotent neural stem cell line. The cells were transfected with a Nurr1 expression vector, and then cocultured with VM type 1 astrocytes. Over 80% of the cells obtained were claimed to have a phenotype resembling endogenous dopaminergic neurons.

Ding et al. (2006) Angewandte Chemie. 118(4):605-607 reports the differentiation of neural progenitor cells into nerve cells using a small molecule neuropathiazol. These cells were further reported to restore damaged tissues and facilitate the growth of intrinsic nerve cells in an animal model of nervous-system diseases. Shetty et al. (2007) Stem Cells 25(8):2014-2017.

However, none of the foregoing patents, patent publications and/or technical literature teach how to orientate neuron growth during the process of differentiation. Neuron cells differentiated from stem cells extend axons in all directions on normal culture surface making the normal cell culture surface unsuitable for regeneration of neurons.

Therefore, a need exists for methods for differentiation of stem cells into oriented neurons. This invention satisfies this need and provides related advantages as well.

### SUMMARY OF THE INVENTION

This invention provides an economical device, method and composition to guide cell growth and differentiation, e.g., neuron growth with a high level of reliability. As compared to chemical treatment, the invention provides more stable, cheaper, long-term guiding effects.

In one aspect, this invention provides a method for growing a population of cells in a substantially parallel orientation, comprising or alternatively consisting essentially of or yet further consisting of contacting at least one isolated cell with a device, wherein the device comprises a plurality of continuously orientating substantially parallel structures on the surface of the device and culturing the cell under conditions that favor division of the at least one cell into a population of cells, thereby growing a population of cells in a substantially parallel orientation. The cell population produced by this method as well as compositions containing them, are also provided herein.

The method utilizes a tissue culture device for aligning cell growth, comprising, or alternatively consisting essentially of or yet further consisting of a plurality of continuously orientating substantially parallel structures on the surface of the device, wherein the orienting structures of the device have an average height of from about 100 nanometers to about 5 micrometers, an average width about 100 nanometers to about 5 micrometers and an average length of from about 1 micrometer to about 20 micrometers. The overall dimension of the device can range from 1 square millimeter to 15 square centimeters, and all variations in between. For example, the device can be in any appropriate overall shape: circular, oval, square or rectangular as long as the orienting, substantially parallel structures on the surface of the device are maintained to achieve the purpose to guide growth and/or differentiation of the cells.

Yet further provided is a method for assaying a potential agent for the ability to affect cell migration, growth and/or differentiation of an isolated stem cell, comprising or alternatively consisting essentially of or yet further consisting of the steps of placing a cell with an agent in the device described above, and allowing the cell to migrate or divide on the surface thereby assaying for the agent's effect on the cell's migration, growth and/or differentiation.

Kits are also provided by this invention. The kits are for use in preparing a population of substantially parallel cells comprising or alternatively consisting essentially of or yet further consisting of a device as described above and instructions to prepare the cell population.

### DESCRIPTION OF THE FIGURES

FIG. 1 shows a device having average dimensions of 1 inch by 2.5 inch in a petri dish.

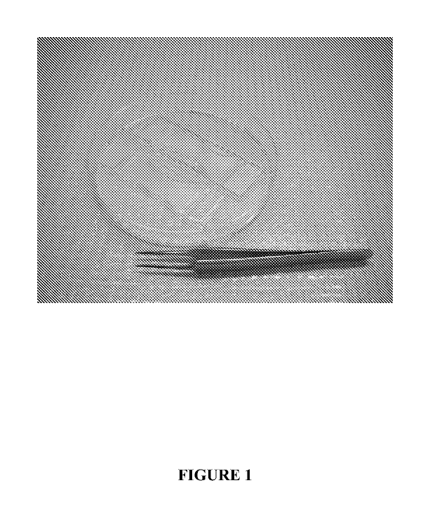

FIG. 2 shows neuron cells differentiated from an Embryoid Body (EB) and grown in a substantially parallel orientation using the device and methods of this invention.

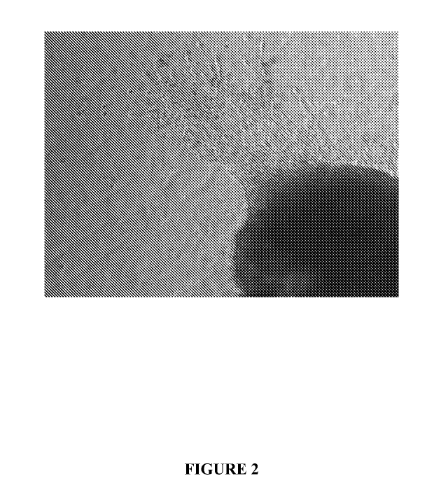

FIG. 3 shows fluorescence imaging of beta (β)-tubulin staining showing the aligned neuron cells grown on the textured surface.

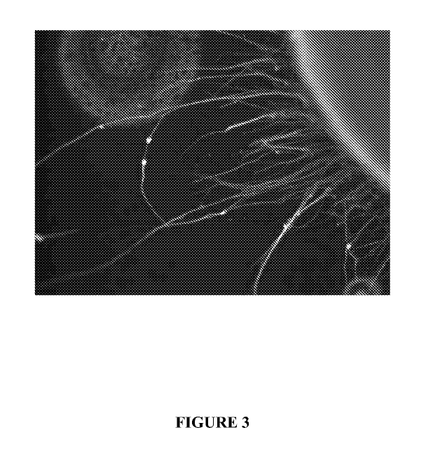

FIG. 4 is a schematic of the device and its use to differentiate EBs or single stem cells.

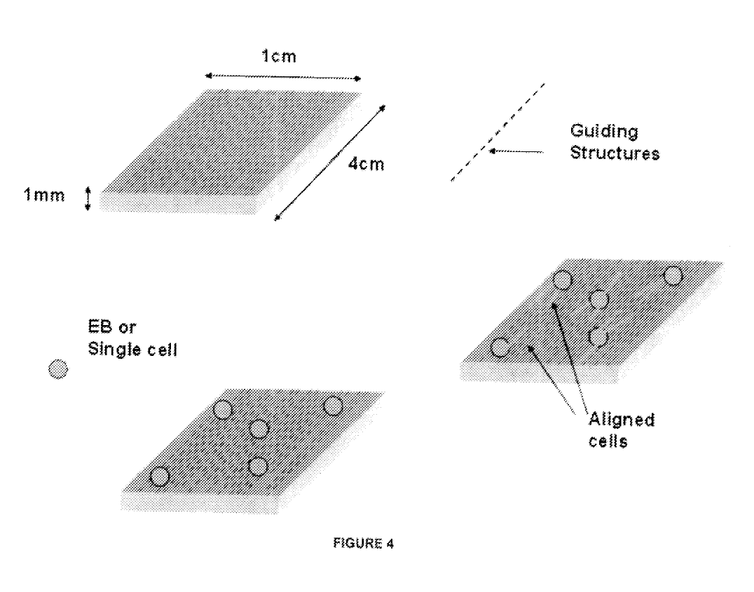

FIG. 5 shows differentiated neural cells plated on a flat substrate showed random growing directions. Scale bar is 200 μm.

FIG. 6 shows differentiated neural cells plated on specific orientated structure substrate showed specific growing direction along the designed structures.

FIG. 7 is a graph depicting align angle analysis of differentiated neural cells on different substrates. Sampling size are larger than 20 cells in each group.

FIG. 8 is a graph showing circularity of differentiated neural cells on different substrates. Sampling size are larger than 20 cells in each group.

### DETAILED DESCRIPTION OF THE INVENTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; Ausubel et al. eds. (1987) Current Protocols In Molecular Biology; the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “device” intends the substrate upon which the orienting structures are constructed and upon which the cells grow. Examples of suitable materials for the device include without limitation: polydimethylsiloxane (PDMS), polyacrylic acid (PAA), poly-meth-acrylic acid (PMAA), ceramics, silica, silica-based materials, polystyrene, polystyrene latex, polyvinyl chloride, polyvinylidene fluoride, polyvinyl acetate, polyvinyl pyrrolidone, polyacrylonitrile, polyacrylamide, polymethyl methacrylate, polytetrafluoroethylene, alginate, polyethylene, polypropylene, and polycarbonate, divinylbenzene styrene-based polymers, celluloses (such as nitrocellulose), cellulosic polymers, polysaccharides, and metals.

A “solution” is intended to refer to a substantially homogeneous mixture of a solute, such as a solid, liquid, or gaseous substance, with a solvent, which is typically a liquid. The solution can be either aqueous or non-aqueous. Examples of suitable solutes in solutions include fluorescent dyes, biological compounds, such as proteins, DNA and plasma, and soluble chemical compounds. Examples of suitable solids include beads, such as polystyrene beads, and powders, such as a metal powder. A “suspension” is intended to refer to a substantially heterogeneous fluid containing a solid, wherein the solid is dispersed throughout the liquid, but does not substantially dissolve. The solid particles in a suspension will typically settle as the particle size is large, compared to a colloid, where the particle size is small such that the suspension does not settle. Examples of suitable suspensions include biological suspensions such as whole blood, cell compositions, or other cell containing mixtures. It is contemplated that any solution, solid or suspension can be mixed using the mixers disclosed herein, provided that the solid has a particle size sufficiently small to move throughout the channels in the mixer.

A “thermoplastic material” is intended to mean a plastic material which shrinks upon heating. In one aspect, the thermoplastic materials are those which shrink uniformly without distortion. A “Shrinky-Dink” is a commercial thermoplastic which is used a children's toy. The shrinking can be either bi-axially (isotropic) or uni-axial (anisotropic). Suitable thermoplastic materials for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC) and spectralon.

In general, the image-forming material is one which is compressed upon heating, bonds to the plastic and is durable (can be used as a mold for multiple iterations). For example, “image-forming material” is, in one aspect, intended to mean a composition, typically a liquid, containing various pigments and/or dyes used for coloring a surface to produce an image or text such as ink and printer toner. In addition to an ink, the image forming material can be a metal, such as gold, titanium, silver, a protein, a colloid, a dielectric substance, a paste or any other suitable metal or combination thereof. Examples of suitable proteins include biotin, fibronectin and collagen. Examples of suitable colloids include pigmented ink, paints and other systems involving small particles of one substance suspended in another. Examples of suitable dielectric substances include metal oxides, such as aluminum oxide, titanium dioxide and silicon dioxide. Examples of suitable pastes include conductive pastes such as silver pastes.

The image forming material can be applied to the thermoplastic material by a variety of methods known to one skilled in the art, such as printing, sputtering and evaporating. The term “evaporating” is intended to mean thermal evaporation, which is a physical vapor deposition method to deposit a thin film of metal on the surface of a substrate. By heating a metal in a vacuum chamber to a hot enough temperature, the vapor pressure of the metal becomes significant and the metal evaporated. It recondenses on the target substrate. As used herein, the term “sputtering” is intended to mean a physical vapor deposition method where atoms in the target material are ejected into the gas phase by high-energy ions and then land on the substrate to create the thin film of metal. Such methods are well known in the art (Bowden et al. (1998) Nature (London) 393:146-149; Bowden et al. (1999) Appl. Phys. Lett. 75:2557-2559; Yoo et al. (2002) Adv. Mater. 14:1383-1387; Huck et al. (2000) Langmuir 16:3497-3501; Watanabe et al. (2004) J. Polym. Sci. Part B: Polym. Phys. 42:2460-2466; Volynskii et al. (2000) J. Mater. Sci. 35:547-554; Stafford et al. (2004) Nature Mater. 3:545-550; Watanabe et al. (2005) J. Polym. Sci. Part B: Polym. Phys. 43:1532-1537; Lacour et al. (2003) Appl. Phys. Lett. 82:2404-2406.)

Another method for applying the image forming material includes, for example “micro-contact printing”. The term “micro-contact printing” refers to the use of the relief patterns on a PDMS stamp to form patterns of self-assembled monolayers (SAMs) of an image-forming material on the surface of a thermoplastic material through conformal contact. Micro-contact printing differs from other printing methods, like inkjet printing or 3D printing, in the use of self-assembly (especially, the use of SAMs) to form micro patterns and microstructures of various image-forming materials. Such methods are well known in the art (Cracauer et al., U.S. Pat. No. 6,981,445; Fujihira et al., U.S. Pat. No. 6,868,786; Hall et al., U.S. Pat. No. 6,792,856; Maracas et al., U.S. Pat. No. 5,937,758).

“Soft-lithography” is intended to refer to a technique commonly known in the art. Soft-lithography uses a patterning device, such as a stamp, a mold or mask, having a transfer surface comprising a well defined pattern in conjunction with a receptive or conformable material to receive the transferred pattern. Microsized and nanosized structures are formed by material processing involving conformal contact on a molecular scale between the substrate and the transfer surface of the patterning device.

The term “receptive material” is intended to refer to a material which is capable of receiving a transferred pattern. In certain embodiments, the receptive material is a conformable material such as those typically used in soft lithography comprise of elastomeric materials, such as polydimethylsiloxane (PDMS). The thermoplastic receptive material, or thermoplastic material, is also a receptive material as it can be etched, for example.

“Imprint lithography” is intended to refer to a technique commonly known in the art. “Imprint lithography” typically refers to a three-dimensional patterning method which utilizes a patterning device, such as a stamp, a mold or mask.

A “mold” is intended to mean an imprint lithographic mold.

A “patterning device” is intended to be broadly interpreted as referring to a device that can be used to convey a patterned cross-section, corresponding to a pattern that is to be created in a target portion of the substrate.

A “pattern” is intended to mean a mark or design.

“Bonded” is intended to mean a fabrication process that joins materials, usually metals or thermoplastics, by causing coalescence. This is often done by melting the materials to form a pool of molten material that cools to become a strong joint, with pressure sometimes used in conjunction with heat, or by itself, to produce the bond.

The term “substantially parallel” shall mean at least 70% parallel, or at least 75% parallel, or at least 80% parallel, or at least 85% parallel, at least 90% parallel, or at least 95% parallel, or at least 97% parallel or alternatively at least 98% parallel.

The term “continuously orienting substantially parallel structures” intends that the structures are fabricated side-by-side and there are no obvious barriers between those structured textures.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and is meant to encompass both cultured and engineered cells or tissues.

As used herein, the “lineage” of a cell defines the heredity of the cell, i.e. its predecessors and progeny. The lineage of a cell places the cell within a hereditary scheme of development and differentiation.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, “stem cell” defines an adult undifferentiated cell that can produce itself and a further differentiated progeny cell and under certain situations, give rise to specialized cells. At this time and for convenience, stem cells are categorized as somatic (adult), embryonic or induced pluripotent stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or H9 (also know as WA01) cell line available from WiCell, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An -induced pluripotent stem cell (iPSC) is an artificially derived stem cell from a non-pluripotent cell, typically an adult somatic cell, produced by inducing expression of one or more stem cell specific genes.

“Embryoid bodies or EBs” are three-dimensional (3-D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) PNAS 100(Suppl. 1)11911-6.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of neuronal progenitor cells or neuronal cells.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) Brain Res. 1091(1):258-264; Pluchino et al. (2005) Brain Res. Brain Res. Rev. 48(2):211-219; and Goh et al. (2003) Stem Cell Res. 12(6):671-679. Neural stem cells can be identified and isolated by neural stem cell specific markers including, but limited to, CD133, ICAM-1, MCAM, CXCR4 and Notch 1. Neural stem cells can be isolated from animal or human by neural stem cell specific markers with methods known in the art. See, e.g., Yoshida et al. (2006) Stem Cells 24(12):2714-22.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell include, without limitation, a progenitor nerve cell.

A “neural precursor cell”, “neural progenitor cell” or “NP cell” refers to a cell that has a capacity to differentiate into a neural cell or neuron. A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue (see, e.g., Vindigni et al. (2009) Neurol. Res. 2009 Aug. 5. [Epub ahead of print]) and adult skin (see, e.g., Joannides (2004) Lancet. 364(9429):172-8). Neural precursor cells can also be derived from stem cells or cell lines or neural stem cells or cell lines. See generally, e.g., U.S. Patent Application Publications Nos.: 2009/0263901, 2009/0263360 and 2009/0258421.

A nerve cell that is “terminally differentiated” refers to a nerve cell that does not undergo further differentiation in its native state without treatment or external manipulation. In one embodiment, a terminally differentiated cell is a cell that has lost the ability to further differentiate into a specialized cell type or phenotype.

A population of cells intends a collection of more than one cell that is identical (clonal) or non-identical in phenotype and/or genotype.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include biocompatible scaffolds, pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

In addition, the image forming material can be applied to the thermoplastic material using “pattern transfer”. The term “pattern transfer” refers to the process of contacting an image-forming device, such as a mold or stamp, containing the desired pattern with an image-forming material to the thermoplastic material. After releasing the mold, the pattern is transferred to the thermoplastic material. In general, high aspect ratio pattern and sub-nanometer patterns have been demonstrated. Such methods are well known in the art (Sakurai et al., U.S. Pat. No. 7,412,926; Peterman et al., U.S. Pat. No. 7,382,449; Nakamura et al., U.S. Pat. No. 7,362,524; Tamada, U.S. Pat. No. 6,869,735).

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional proviso that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

An “effective amount” is an amount sufficient to effect beneficial or desired results whether it is therapeutic or diagnostic. An effective amount can be administered in one or more administrations, applications or dosages.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell, it is preferable to use a positive control (a sample with an aspect that is known to affect differentiation) and a negative control (an agent known to not have an affect or a sample with no agent added).

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

The term patient or subject refers to animals, including mammals, preferably humans, who are treated with the pharmaceutical compositions or in accordance with the methods described herein.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

A neuron is an excitable cell in the nervous system that processes and transmits information by electrochemical signaling. Neurons are found in the brain, the vertebrate spinal cord, the invertebrate ventral nerve cord and the peripheral nerves. Neurons can be identified by a number of markers that are listed on-line through the National Institute of Health at the following website: “stemcells.nih.gov/info/scireport/appendixe.asp#eii,” and are commercially available through Chemicon (now a part of Millipore, Temecula, Calif.) or Invitrogen (Carlsbad, Calif.). For example, neurons may be identified by expression of neuronal markers B-tubulin III (neuron marker, Millipore, Chemicon), Tuj1 (beta-III-tubulin); MAP-2 (microtubule associated protein 2, other MAP genes such as MAP-1 or -5 may also be used); anti-axonal growth clones; ChAT (choline acetyltransferase (motoneuron marker, Millipore, Chemicon); Olig2 (motorneuron marker, Millipore, Chemicon), Olig2 (Millipore, Chemicon), CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; alpha-internexin; NeuN (neuron-specific nuclear protein); NF (neurofilament); NGF (nerve growth factor); gamma-NSE (neuron specific enolase); peripherin; PH8; PGP (protein gene product); SERT (serotonin transporter); synapsin; Tau (neurofibrillary tangle protein); anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), VGLUT (vesicular glutamate transporter).

The term neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

Other neurodegenerative conditions include dementias, regardless of underlying etiology, including age-related dementia and other dementias and conditions with memory loss including dementia associated with Alzheimer's disease, vascular dementia, diffuse white matter disease (Binswanger's disease), dementia of endocrine or metabolic origin, dementia of head trauma and diffuse brain damage, dementia pugilistica and frontal lobe dementia.

The term treating (or treatment of) a neurodegenerative disorder or condition refers to ameliorating the effects of, or delaying, halting or reversing the progress of, or delaying or preventing the onset of, a neurodegenerative condition as defined herein.

### Methods for Preparing Wrinkles and Aligning Cells

In one aspect, the present invention provides a tissue culture device for aligning cell growth, comprising a plurality of continuously orientating substantially parallel structures on the surface of the device, wherein the orienting structures of the plurality have an average height of from about 100 nanometers to about 5 micrometers, an average width in the range of 100 nanometers to 5 micrometers and an average length of from about 1 micrometer to about 20 micrometers.

In another aspect, the orienting structures of the plurality have an average height selected from the group of about 100 nanometers, about 200 nanometers, about 300 nanometers, about 500 nanometers, about 700 nanometers, about 1 micrometer, about 2 micrometers, about 3 micrometers, about 4 micrometers, or about 5 micrometers. In a further aspect, the orienting structures of the plurality have an average width of about 100 nanometers, about 200 nanometers, about 300 nanometers, about 500 nanometers, about 700 nanometers, about 1 micrometer, about 2 micrometers, about 3 micrometers, about 4 micrometers, or about 5 micrometers. In a yet further aspect, the orienting structures of the plurality have an average length of about 1 micrometer, about 2 micrometers, about 5 micrometers, about 7 micrometers, about 10 micrometers, about 12, micrometers, about 15 micrometers, about 17 micrometers, about 19 micrometers, and about 20 micrometers.

The overall dimension of the device can range from 1 square millimeter to 15 square centimeters, and all variations in between. For example, the device can be in any appropriate overall shape, circular, oval, square or rectangular as long as the orienting, substantially parallel structures on the surface of the device are maintained to achieve the purpose to guide growth and/or differentiation of the cells.

The device can be constructed from a variety of materials, which include but are not limited to polydimethylsiloxane (PDMS), polyacrylic acid (PAA), poly-meth-acrylic acid (PMAA), gelatin, alginate, agarose, polyethylene glycol, cellulose nitrate, polyacrylamide, or chitosan. In a specific aspect, the device comprises polydimethylsiloxane (PDMS).

This invention provides a method for growing a population of cells in a substantially parallel orientation, comprising, or alternatively consisting essentially of, or yet further consisting of, contacting at least one isolated cell with a device, wherein the device comprises, or alternatively consists essentially of, or yet consists of, a plurality of continuously orientating substantially parallel structures on the surface of the device, and culturing the cell under conditions that favor division of the at least one cell into a population of cells, thereby growing the population of cells in a substantially parallel orientation.

In one aspect, the orienting structures of the device have an average height of from about 100 nanometers to about 5 micrometers, an average width of about 100 nanometers to about 5 micrometers and an average length of from about 1 micrometer to about 20 micrometers.

In another aspect, the orienting structures of the device have an average height selected from of about 100 nanometers, about 200 nanometers, about 300 nanometers, about 500 nanometers, about 700 nanometers, about 1 micrometer, about 2 micrometers, about 3 micrometers, about 4 micrometers, or about 5 micrometers.

In a further aspect, the orienting structures of the plurality have an average width of about 100 nanometers, about 200 nanometers, about 300 nanometers, about 500 nanometers, about 700 nanometers, about 1 micrometer, about 2 micrometers, about 3 micrometers, about 4 micrometers, or about 5 micrometers.

In a yet further aspect, the orienting structures of the device have an average length of about 1 micrometer, about 2 micrometers, about 5 micrometers, about 7 micrometers, about 10 micrometers, about 12, micrometers, about 15 micrometers, about 17 micrometers, about 19 micrometers, or about 20 micrometers.

In a yet further aspect, the device comprises, or alternatively consists essentially of, or yet further consists of one or more material of the group polydimethylsiloxane, poly-meth-acrylic acid (PMAA), alginate and polyacrylic acid (PAA). In a particular aspect, the device comprises, or alternatively consists essentially of, or yet further consists of, polydimethylsiloxane.

Various types of cell populations may be grown or aligned on the surface of the present invention. In one aspect, the cell is an isolated prokaryotic or eukaryotic cell. In another aspect, the cell is an isolated eukaryotic cell. The isolated eukaryotic cell may be of animal origin, e.g., a mammalian cell, a simian cell, a bovine cell or a murine cell.

In one aspect, the isolated eukaryotic cell may be an isolated stem cell. In some embodiments, the isolated stem cell is selected from the group consisting of an embryonic stem cell, a pluriopotent stem cell, a parthenogenetic stem cell, a somatic stem cell and an induced pluripotent stem cell (iPS stem cell). Methods to isolate, grow and culture such cells are known in the art. See for example, US Patent Publ. Nos. 2009/0081784; 2009/0075374; 2009/0068742; and 2009/0047263.

In one aspect, the isolated stem cell is of animal origin. In some embodiments, the animal origin is mammalian, simian, bovine, ovine, or murine. In one aspect, the mammalian origin is human.

In another aspect, the isolated eukaryotic cell is a neural stem cell, a neural precursor cell, fetal or neonatal cell.

The cells are grown under typical conditions, e.g., temperature, pressure and atmosphere as known to those of skill in the art. It is the substrate upon which the cells are differentiated that allows the unique orientation of the cells, i.e., in a substantially parallel orientation.

The device is particularly suited to expand and grow neural stem cells from isolated or populations of isolated embryonic stem cells, embryoid bodies, iPSCs, parthenogenetic stem cells, or neural stem cells to population of neural cells which are orientated substantially parallel to each other. The present invention differs from prior art devices such as that described in Mata et al. (2007) Int. J. Nanomedicine 2(3):389-406. Mata et al. (2007) describes a minchannel device for growing connective tissue progenitors which require the cells to grow in the channels which causes the cells to pack tightly in the channel and stop proliferating after several days. Moreover, due to the physical boundaries of the of the channel walls, those cells can't connect to the cells grown in other channels. Applicants' device, in contrast, grows the cells on top of the aligning structures and provides the cues of growing direction to the cells, but does not limit their growth.

Also provided is a method for assaying a potential agent for the ability to affect cell migration, growth and/or differentiation of an isolated stem cell, comprising, or alternatively consisting essentially of, or yet further consisting of the steps of placing a cell with an agent in a device as described herein and allowing the cell to migrate or divide in the device assaying for the agent's effect on the cell's migration, growth and/or differentiation. Suitable positive and negative controls can be cultured for the purpose of comparison. Assaying can be accomplished by any method known to those of skill in the art, e.g., visual observation with the naked eye or under a microscope.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides and synthetic organic compounds based on various core structures; these compounds are also included in the term “agent”. In addition, various natural sources can provide compounds for screening, such as plant or animal extracts and the like. It should be understood, although not always explicitly stated that the agent is used alone or in combination with another agent, having the same or different biological activity as the agents identified by the inventive screen. The agents and methods also are intended to be combined with other therapies.

When the agent is a nucleic acid, it can be added to the cell cultures by methods known in the art, which includes, but is not limited to calcium phosphate precipitation, microinjection or electroporation. Alternatively or additionally, the nucleic acid can be incorporated into an expression or insertion vector for incorporation into the cells. Vectors that contain both a promoter and a cloning site into which a polynucleotide can be operatively linked are well known in the art and briefly described infra.

When the agent is a composition other than a DNA or RNA nucleic acid molecule, the suitable conditions comprise directly added to the cell culture or added to culture medium for addition. As is apparent to those skilled in the art, an “effective” amount must be added which can be empirically determined and must be administered for an effective amount of time.

This invention also provides the isolated population of substantially parallel cells, e.g., neurons prepared by the method of this invention as well as a composition comprising, or alternatively consisting essentially of, or yet further consisting of, the population of cells described above and a carrier, e.g., a pharmaceutically acceptable carrier. The population of cells may be substantially homogenous or substantially heterogeneous, depending on the cell of origin and culture conditions. In one aspect, the population of cells are differentiated from an embryoid body. A population of cells intends a plurality of cells of at least about 1×10⁶ cells, or alternatively at least 5×10⁵ cells, or alternatively at least 1×10⁵ cells. The population of cells can be further isolated or separated from the device for further diagnostic or therapeutic application.

It is considered a common practice to use either one of the following methods to remove cells from the surface: (1) trypsin-EDTA solution for enzymatic cleavage of extracellular matrix; (2) cell dissociation buffer (enzyme-free) Hanks' based solution; and (3) use of a cell scrapper for mechanical removal of cells. These methods are known in the art and publicly available at the web address wicell.org/index.php?options=com (last accessed on Aug. 25, 2010)(published procedures include, e.g., SOP-CC-011A_Collagenase; SOP-CC-037A_alternate splitting.doc; SOP-CC-025A_selection and maintenance.doc).

The cells are useful in diagnostic and therapeutic applications as known to those of skill in the art. Accordingly, this invention also provides a method for treating a disease or disorder requiring substantially parallel cells, such as a neurological condition, by administering to a subject in need of the treatment an effective amount of the cells of this invention. Such disorders, include but are not limited to neurodegenerative disorders as defined herein, such as Traumatic Brain Injury, Alzheimer's disease, Parkinson's disease, epilepsy, Huntington's disease, and stroke. Cell populations that can reconstitute the neural network is key to realizing these therapies. The methods are accomplished by administering an effective amount of the cells. Modes of administration and the amount of cells necessary for the specific treatment. The cells may be autologous or allergenic to the subject receiving treatment.

In certain embodiments, the cells are administered with at least one other cell type, such as an astrocyte, oligodendrocyte, neuron, neural progenitor or other cell. Alternatively or in addition, the cells are administered with at least one other agent, such as a drug for neural therapy, or another beneficial adjunctive agent such as an anti-inflammatory agent, anti-apoptotic agents, antioxidant or growth factor. In these embodiments, the other agent can be administered simultaneously with, or before, or after, the neurons.

In certain embodiments, the cells are administered at a pre-determined site in the central or peripheral nervous system of the patient. They can be administered by injection or infusion, or encapsulated within an implantable device, or by implantation of a matrix or scaffold containing the cells.

In certain embodiments, the pharmaceutical composition is formulated for administration by injection or infusion. Alternatively, it may comprise an implantable device in which the cells are encapsulated, or a matrix or scaffold containing the cells.

According to yet another aspect of the invention, a kit is provided for treating a patient having a neurodegenerative condition. The kit comprises a pharmaceutically acceptable carrier, a population of the above-described cells and instructions for using the kit in a method of treating the patient. The kit may further comprises at least one reagent and instructions for culturing the cells. It can also comprise, or alternatively consisting of, or yet further consisting of, a population of at least one other cell type, or at least one other agent for treating a neurodegenerative condition.

### EXAMPLES

The present technology is further understood by reference to the following example. The present technology is not limited in scope by the examples, which are intended as illustrations of aspects of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### Example 1

To create the device and orientation guiding structures, pre-stressed thermoplastic sheets are used in the method described in U.S. Ser. No. 61/161,738, filed Mar. 19, 2009 and PCT Appl. No. PCT/US2008/083283, each incorporated herein by reference. To make the device, a metal as an image forming material is deposited onto a pre-stressed thermoplastic material and then the thermoplastic material is reduced by at least about 60% and then the surface is prepared via lithography.

The thermoplastic material onto which the metal is deposited is a heat sensitive thermoplastic receptive material. In certain embodiments, the depositing of heat sensitive thermoplastic receptive material is by evaporating, which is a physical vapor deposition method to deposit a thin film of metal on the surface of a substrate. By heating a metal in a vacuum chamber to a hot enough temperature, the vapor pressure of the metal becomes significant and the metal evaporated. It recondenses on the target substrate. The height of the metal is dependent on length of processing time. The thermoplastic substrate must be far enough from the source such that the plastic does not heat up during deposition.

After the metal is deposited on the thermoplastic, it is placed in an oven, or similar device, to be heated, and upon heating, because of the stiffness incompatibility between the metal and the shrinking thermoplastic, wrinkles form. The spacing between the metal wrinkles can be controlled by the amount of heating, and hence shrinkage.

Wrinkle height can be controlled by adjusting the metal film thickness. FIG. 17 of the PCT application PCT/US2008/083283 shows a plot of the maximum average wrinkle height as a function of metal layer thickness. Therefore, one can easily predict the spacing between and height of the metal wrinkles by adjusting the thickness of metal deposited onto the thermoplastic material and the time the thermoplastic material is heated. The thickness of metal deposited onto the thermoplastic material can be easily controlled using the metal deposition methods disclosed herein by adjusting parameters such as time, temperature, and the like. Such methods are well known to one of skill in the art.

To create the orientation guiding structures, the thermoplastic sheet to shrink in uni-axis, the thermoplastic material is restricted by mechanical clamps. The orientation structures mold is fabricated on the top surface of the sheet. The dimensions of structures range from 100 nm to 20 μm. Here, the properties of guiding structures are controlled by changing heating temperature, heat duration, deposited metal thickness, and the length-width ration of thermoplastic sheets. Using those metal structures as mold, the structures can be transferred by methods such as lithography onto different biocompatible materials, such as PDMS, PAA, PLA.

To grow the cells, human embryonic stem cell colonies were treated with dispase (0.5 mg/ml, Invitrogen) to remove colonies from MEF feeder layers. The colonies were cultured in ultra-low attachment dish (Costar) for 4 days in N₂ medium consisting of DMEM/F12, nonessential amino acids, sodium pyruvate (Invitrogen), N₂ supplement (Invitrogen), and FGF-2 (8 ng/ml) to form Embryoid Bodies (EBs). At day 5, EBs were attached by using N₂ medium supplement with laminin (1 lg/ml, Invitrogen) on the orientation guiding structures. Cells were then cultured in laminin-contained N₂ medium for another 7 days.

### Example 2

In the following experiments, cell culture and orientated substrates fabrication were followed same processes as mentioned.

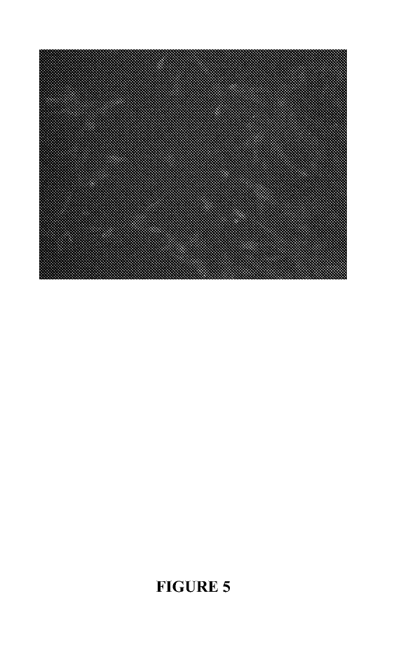

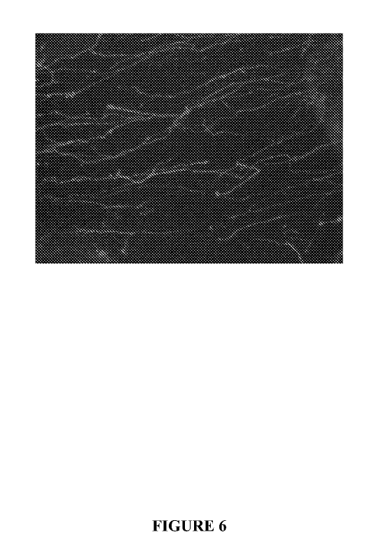

Immunofluorescence was used to determine non-neural lineage cells and neural lineage cells. In FIGS. 5 and 6, neural cells differentiated from hESCs were stained with neuron-specific marker β-tubulin III. Images were acquired with fluorescent microscope and were analyzed with image analysis software (NIS-Elements, Nikon.)

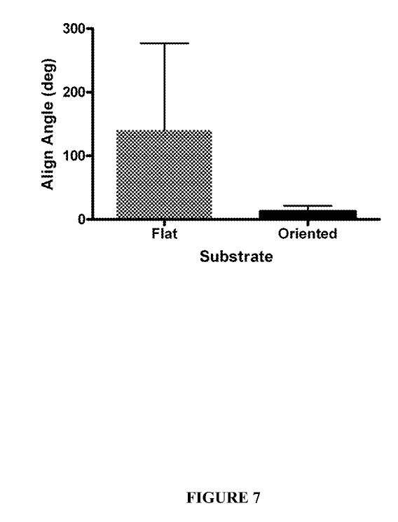

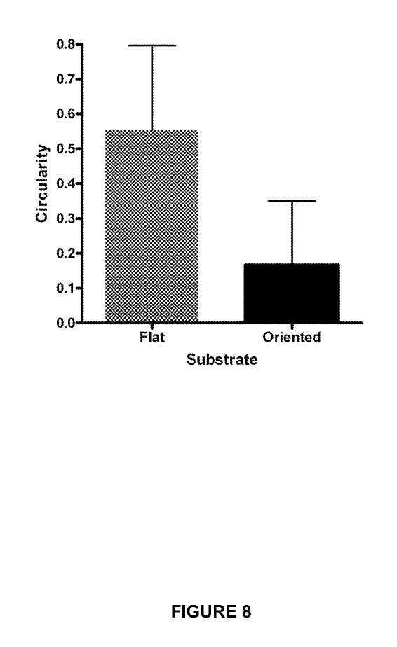

In the following analysis (FIGS. 7 and 8), undifferentiated cells were not be sampled and only cells expressed β-tubulin III were analyzed. In FIG. 7, Applicants used align angle of cells as an indicator of cellular growing orientation. First, Applicants defined a reference direction on each substrate. The reference direction was set randomly on the flat substrate, and the reference direction on oriented structure was set parallel to those surface structures.

Applicants then measured the angles between the ref and cell's long-axis. Results showed the angle was 140±136° on flat substrate, and 12.5±8.8° on oriented substrate. It demonstrated that cells grew on oriented substrate were highly aligned in specific direction, which parallel to the designed pattern.

Circularity was used as another indication for cell growing orientation. If cells spread into random directions, the circularity of each cell is close to one. In contrast, lower circularity may indicate cell spread into more specific direction. Data showed circularity was 0.55±0.24 on flat substrate, and 0.16±0.18 on oriented structure.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for growing a population of cells in a substantially parallel orientation, comprising contacting at least one isolated cell with a device, wherein the device comprises a plurality of continuously orientating substantially parallel structures on the surface of the device, and culturing the cell under conditions that favor division of the at least one cell into a population of cells, thereby growing the population of cells in a substantially parallel orientation.

2. The method of claim 1, wherein the orienting structures of the device have an average height of from about 100 nanometers to about 5 micrometers, an average width of about 100 nanometers to about 5 micrometers and an average length of from about 1 micrometer to about 20 micrometers.

3. The method of claim 1, wherein the orienting structures of the device have an average height selected from the group consisting of about 100 nanometers, about 200 nanometers, about 300 nanometers, about 500 nanometers, about 700 nanometers, about 1 micrometer, about 2 micrometers, about 3 micrometers, about 4 micrometers, or about 5 micrometers.

4. The method of claim 1, wherein the orienting structures of the device have an average width of about 100 nanometers, about 200 nanometers, about 300 nanometers, about 500 nanometers, about 700 nanometers, about 1 micrometer, about 2 micrometers, about 3 micrometers, about 4 micrometers, or about 5 micrometers.

5. The method of claim 1, wherein the orienting structures of the device have an average length of about 1 micrometer, about 2 micrometers, about 5 micrometers, about 7 micrometers, about 10 micrometers, about 12, micrometers, about 15 micrometers, about 17 micrometers, about 19 micrometers, or about 20 micrometers.

6. The method of claim 1, wherein the device comprises one or more material of the group of polydimethylsiloxane, poly-meth-acrylic acid (PMAA), alginate or polyacrylic acid (PAA).

7. The method of claim 1, wherein the device comprises polydimethylsiloxane.

8. The method of claim 1, wherein the isolated cell is an isolated eukaryotic cell or an isolated prokaryotic cell.

9. The method of claim 1, wherein the isolated cell is an isolated stem cell.

10. The method of claim 9, wherein the isolated stem cell is selected from the group of an embryonic stem cell, a pluriopotent stem cell, a parthenogenetic stem cell, a somatic stem cell or an iPS stem cell.

11. The method of claim 1, wherein the isolated cell is selected from the group of a mammalian cell, a simian cell, a bovine cell or a murine cell.

12. The method of claim 1, wherein the isolated cell is a human cell.

13. The method of claim 1, wherein the isolated cell is cultured under conditions that favor the preparation of a population of neural cells and wherein the cells of the population are substantially parallel to each other.

14. The method of claim 1, further comprising removing the population of cells from the device.

15. The population of cells prepared by the method of claim 14.

16. The population of claim 15, further comprising an endogenous polynucleotide or polypeptide.

17. The population of claim 16, further comprising a carrier or a biological scaffold.

18. A method for treating a neurodegenerative disorder comprising administering to a subject in need of such treatment an effective amount of the population of 16 or 17.

19. A method for assaying a potential agent for the ability to affect cell migration, growth and/or differentiation of an isolated stem cell, comprising contacting at least one isolated stem cell with a device and with the agent, wherein the device comprises a plurality of continuously orientating substantially parallel structures on the surface of the device, and culturing the cell under conditions that favor division of the at least one cell into a population of cells and observing the cell growth, thereby assaying for the agent's effect on the cell's migration, growth and/or differentiation.

20. A kit for use in preparing a population of substantially parallel cells comprising a device comprising a plurality of continuously orientating substantially parallel structures on the surface and instructions for use of the device.

21. An isolated population of substantially parallel neurons.

22. The population of claim 21, wherein the neurons are differentiated from an embryoid body.

23. A composition comprising the population of claim 21 and a carrier.

24. The composition of claim 23, wherein the carrier is a pharmaceutically acceptable carrier.
